(19) United States
(12) Patent Application Publication
(10) Pub. No.: US 2002/0071820 A1
(43) Pub. Date: Jun. 13, 2002

(54) Use of cubic gel particles as an anti-pollution agent, in particular in a cosmetic composition

(75) Inventors: Isabelle Afriat, New York, NY (US); Bruno Biatry, Vincennes (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'Oreal, Paris (FR)

(21) Appl. No.: 09/875,993

(22) Filed: Jun. 8, 2001

(30) Foreign Application Priority Data
Jun. 8, 2000 (FR) 00-07342

(51) Int. Cl. A61K 7/075; A61K 7/08

(52) U.S. Cl. 424/70.31

(57) ABSTRACT

The present application relates to the use in topical application of cubic gel particles as an antipollution agent, in particular as an anti-pollution cosmetic agent. The application also relates to a treatment process for protecting the body against the effects of pollution, which includes applying to the keratin material a composition containing an effective amount of cubic gel particles in a physiologically acceptable medium. The cubic gel particles are preferably in aqueous dispersion and are preferably formed either from a compound chosen from 3,7,11,15-tetramethyl-1,2,3-hexadecanetriol, phytanetriol, N-2-alkoxycarbonyl derivatives of N-methylglucamine and unsaturated fatty acid monoglycerides, and from a dispersing and stabilizing agent, or from a mixture of at least two amphiphilic compounds, one of the amphiphilic compounds preferably being capable of forming a lamellar phase in the presence of water and the other preferably being capable of forming an inverse hexagonal phase in the presence of water.

### BACKGROUND OF THE INVENTION

1. Field of the Invention

The present application relates to the use of cubic gel particles as an anti-pollution agent, and also to a cosmetic treatment process for protecting the body against the effects of pollution, which includes applying to the keratin material a composition containing an effective amount of cubic gel particles in a physiologically acceptable medium.

2. Discussion of the Background

Certain urban environments are regularly subjected to peeks of pollution. An individual in his daily environment, and particularly in an urban area, may be subjected to a whole range of factors attacking keratin materials, and in particular, the skin, the scalp and the hair, by various airborne pollutants.

Among the pollutants which may exert deleterious effects on keratin materials, toxic gases such as ozone, carbon monoxide, nitrogen oxides or sulphur oxides are among the major constituents. It has been found that these toxic gases promote the desquamation of keratin materials, making them dull and dirty. Similarly, these gases cause cellular asphyxia of the said keratin materials.

It is moreover known that heavy metals (lead, cadmium and mercury) are atmospheric pollutants whose emissions have increased considerably, especially in urban and industrial environments. In addition to certain toxic effects which they cause, heavy metals have the property of reducing the activity of the cellular defense means against free radicals (see for example R. S. Dwivedi, J. Toxicol. Cut. & Ocular Toxical. 6(3), 183-191 (1987)). Thus, heavy metals aggravate the toxic effects of gaseous pollutants by reducing the efficacy of the natural defense means, and bring about an acceleration of the phenomenon of cell aging. This is particularly true for keratin materials and especially the skin, the scalp and the hair, which are in direct and permanent contact with the external environment.

Thus, the harmful effects of pollution on keratin materials affect cell respiration and are reflected by accelerated aging of the skin, with a dull complexion and the early formation of wrinkles or fine lines, and also by a reduction in the vigor of the hair, which thus acquires a dull appearance. In addition, owing to pollution the skin and the hair become dirty more quickly. Furthermore, pollution may cause allergy phenomena on the skin.

Thus, there is a need for compositions to prevent the harmful effects due to pollutants (gases or heavy metals), so as to protect keratin materials against these external pollutants.

### SUMMARY OF THE INVENTION

One object of the present invention is to protect keratin materials against the harmful effects due to pollutants (preferably gases or heavy metals).

Another object of the present invention is to prevent the harmful effects on keratin materials due to pollutants (preferably gases or heavy metals).

It has now been found, entirely surprisingly, that cubic gel particles, applied to keratin materials, and in particular to the skin, protect these keratin materials against the effects of pollutants found in the atmosphere.

Thus, one subject of the present invention is the cosmetic or dermatological use of cubic gel particles as an antipollution agent, in a composition for topical application to keratin materials.

Another subject of the invention is the use of cubic gel particles to prepare a topical-application composition for protecting keratin materials against the harmful effects of pollution.

Another embodiment of the present invention provides a method for protecting keratin materials from the harmful effects of pollution, which includes topically applying a composition containing an effective amount of cubic gel particles to the keratin materials.

Another embodiment of the present invention provides a treatment process for protecting a keratin material against the effects of pollution, which includes applying to keratin material a composition containing an effective amount of cubic gel particles in a physiologically acceptable medium.

Another embodiment of the present invention provides a treatment process for improving the cell respiration and/or for reducing desquamation and/or for preventing keratin material from becoming dull and/or dirty, which includes applying to the keratin material a composition containing an effective amount of cubic gel particles in a physiologically acceptable medium.

### BRIEF DESCRIPTION OF THE PREFERRED EMBODIMENTS

Various other objects, features and attendant advantages of the present invention will be more fully appreciated as the same becomes better understood from the following detailed description of the preferred embodiments of the invention.

The expression “topical application” means herein an external application to keratin materials, these especially being the skin, the scalp, the eyelashes, the eyebrows, the nails and mucous membranes.

The cubic gel particles may be used alone and may constitute the composition to be used, or may be incorporated into a composition and especially into an oil-in-water (O/W) or water-in-oil (W/O) emulsion.

The expression “effective amount” means an amount which is sufficient to achieve the desired aim. The cubic gel particles used according to the invention may be present in the composition for topical application in an amount ranging, for example, from 0.1% to 20% by weight and preferably from 0.1% to 10% by weight relative to the total weight of the composition. These ranges include all values and subranges therebetween, including 2, 3, 4, 5, 6, 7, 8, 9, 11, 12, 13, 14, 15, 16, 17, 18, and 19%.

The term “cubic gel” denotes transparent gels which are isotropic in polarized light and which are in the form of a cubic liquid crystal phase. The cubic phases are organized in a bipolar manner into distinct hydrophilic and lipophilic domains, in close contact and forming a thermodynamically stable three-dimensional network. Such an organization has been disclosed in particular in Luzzati (1968), “Biological Membranes” (Chapman, D. Ed.), vol. 1, 71-123 and in Mariani et al. (1988), J. Mol. Biol., 204, 165-189, and also in “La Recherche” (1992), vol. 23, 306-315, the entire contents of each of which being hereby incorporated by reference. According to the arrangement of the hydrophilic and lipophilic domains, the cubic phase is said to be of normal or inverse type. The term “cubic gel” used according to the present invention combines, of course, gels with cubic phases of different types.

When cubic gel is dispersed in aqueous medium, cubic gel particles in dispersion are obtained, particles which have the same structure as cubic gel.

Any type of cubic gel may be used according to the present invention. The cubic gel particles used are advantageously in aqueous dispersion. They may be obtained in particular by the two preferred embodiments described below.

According to a first embodiment, the particles are its aqueous dispersion and are formed from a mixture including (i) 0.1% to 15% by weight, relative to the total weight of the composition, of at least one compound chosen from 3,7,11,15-tetramethyl-1,2,3-hexadecanetriol, or phytanetriol, N-2-alkoxycarbonyl derivatives of N-methylglucamine and unsaturated fatty acid monoglycerides, and (ii) 0.05% to 3% by weight, relative to the total weight of the composition, of at least one dispersing and stabilizing agent, the said agent being chosen from surfactants that are water-soluble at room temperature, containing a saturated or unsaturated, linear or branched fatty chain containing from 8 to 22 carbon atoms. The range for (i) includes all values and subranges therebetween, including 0.5, 1, 2, 3, 4, 5, 6, 7, 8, 9, 10, 11, 12, 13, and 14%. The range for (ii) includes all values and subranges therebetween, including 0.1, 1.1, 1.5, 2, 2.5 and 2.75%.

According to this embodiment of the cubic gel particles used according to the invention, the relative weight proportion of compound (i) relative to the weight of the dispersing and stabilizing agent (ii) may range, for example, from 2 to 200 and preferably from 5 to 50. These ranges include all values and subranges therebetween, including 3, 10, 20, 30, 40, 75, 100, 125 and 150.

The phytanetriol which may be used as compound (i) is a known compound, which is sold in particular under the name “Phytanetriol-63926” by the company Roche.

Among the N-2-alkoxycarbonyl derivatives of N-methylglucamine, mention may be made in particular of those corresponding to formula (I) below:

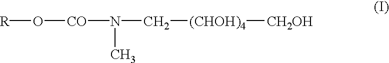

in which R represents a branched alkyl radical containing from 6 to 18 carbon atoms.

Among these derivatives, mention may be made in particular of N-2-hexyldecyloxycarbonyl-N-methylglucamine, N-2-ethylhexyloxycarbonyl-N-methylglucamine and N-2-butyloctyloxycarbonyl-N-methylglucamine, and mixtures thereof.

The compounds of formula (I) as defined above are disclosed and may be prepared according to the process disclosed in EP-A-711 540, which is hereby incorporated in its entirety by reference. This process preferably includes:

(a) dissolving N-methylglucamine in a mixture of water and an organic solvent, the solvent possibly being tetrahydrofuran, for example,

(b) dispersing sodium bicarbonate in the mixture obtained above, in a suitable proportion corresponding to about four times the molar proportion of N-methylglucamine,

(c) then introducing an alkyl chloroformate, the alkyl radical being C₆-C₁₈, into the reaction mixture obtained, in a suitable proportion, generally an equimolar proportion relative to that of N-methylglucamine, and then leaving the mixture to react, and

(d) filtering the reaction mixture obtained after step (c), collecting the pasty residue obtained by filtration and then dissolving it in acetone to crystallize it at a temperature of about 5° C. After filtration, the crystals of the N-2-alkoxycarbonyl derivative of N-methylglucamine formed are spin-filtered and dried under vacuum.

When compounds of formula (I) are used as compound (i), the cubic gel particles used according to the invention preferably contain a mixture of such a compound and of phytanetriol, and more precisely a mixture including an amount of phytanetriol ranging from 1% to 40% by weight and better still from 10% to 30% by weight relative to the weight of the mixture, and an amount of N-2-alkoxycarbonyl derivative of N-methylglucamine of formula (I) ranging from 60% to 99% by weight and better still from 70% to 90% by weight relative to the weight of the mixture. These ranges include all values and subranges therebetween, including 2, 5, 7, 15, 20, 25, 35, 50, 55, 65, 75, 80, 85, and 95% as appropriate for the respective amounts of phytanetriol and N-2-alkoxycarbonyl derivative of N-methylglucamine of formula (I).

The unsaturated fatty acid monoglycerides which may be used as compounds (i) in this first embodiment are preferably those with an unsaturated fatty chain containing from 16 to 22 carbon atoms. Among these monoglycerides, mention may be made in particular of glyceryl monooleate or monooleine and glyceryl monolinoleate or monolinoleine. Needless to say, to prepare the dispersions of cubic gel particles, it is possible to use a mixture of monoglycerides as defined above, and also a mixture of unsaturated fatty acid monoglycerides and of saturated fatty acid monoglycerides, the proportion of saturated fatty acid monoglycerides however preferably being less than that of the unsaturated fatty acid monoglycerides.

When unsaturated fatty acid monoglycerides are used as compound (i), the cubic gel particles preferably contain, as compound (i), a mixture of such a compound and of phytanetriol, and more precisely a mixture including an amount of phytanetriol ranging from 1% to 50% by weight and better still from 10% to 30% by weight relative to the total weight of the mixture and an amount of unsaturated fatty acid monoglyceride ranging from 50% to 99% by weight and better still from 70% to 90% by weight relative to the weight of the mixture. These ranges include all values and subranges therebetween, including 2, 5, 7, 15, 20, 25, 35, 55, 65, 75, 80, 85, and 95% for the respective amounts of phytanetriol and unsaturated fatty acid monoglyceride as appropriate.

The agent (ii) of this first embodiment for dispersing and stabilizing the cubic gel particles is preferably chosen from:

(1) alkyl or alkenyl ethers or esters of a polyol,

(2) N-acyl amino acids and derivatives thereof, and peptides N-acylated with an alkyl or alkenyl radical, and salts thereof,

(3) alkyl or alkenyl ether or ester sulphates, derivatives thereof and salts thereof,

(4) polyoxyethylenated fatty alkyl or alkenyl ethers or esters,

(5) polyoxyethylenated alkyl or alkenyl carboxylic acids and salts thereof,

(6) N-alkyl or alkenyl betaines,

(7) alkyl or alkenyl trimethylammoniums and salts thereof, and

(8) mixtures thereof.

In the compounds listed above, the alkyl and alkenyl radical contain from 8 to 22 carbon atoms and may be in the firm of mixtures.

(1) As alkyl or alkenyl ethers or esters of a polyol, mention may be made in particular of:

(a) sorbitan alkyl or alkenyl esters polyoxyethylenated containing at least 20 ethylene oxide units, such as sorbitan palmitate 20 EO or Polysorbate 40 sold under the name “Montanox 40 DF” by the company SEPPIC, and sorbitan laurate 20 EO or Polysorbate 20 sold under the name “Tween 20” by the company ICI,

(b) oxyethylenated or non-oxyethylenated polyglyceryl alkyl or alkenyl esters including at least 10 units derived from glycerol, such as polyglyceryl-10 laurate sold under the name “Decaglyn 1-L” by the company Nikko Chemicals,

(c) polyglyceryl alkyl or alkenyl ethers, such as polyglyceryl-3 hydroxylauryl ether sold under the name “Chimexane NF” by the company Chimex, and

(d) alkyl or alkenyl esters or ethers of mono- or polysaccharides such as those derived from glucose, fructose, galactose, maltose or lactose, and in particular 1- and 6-monoesters of D-fructose, of decylglucose and of decylpolyglucose.

(2) As N-acyl amino acids and derivatives thereof, and as peptides N-acylated with an alkyl or alkenyl radical, and salts thereof, the ones that are preferably used are those for which the alkyl or alkenyl radical contains at least 12 carbon atoms.

According to the invention, the term “amino acids” means alpha-, beta- or gamma-amino acids. N-acyl amino acid salt which may be mentioned, for example, are the salts of N-acylglutamate, such as monosodium cocoylglutamate, monosodium lauroylglutamate, disodium (C₁₄-C₂₀) alkylglutamate (the C₁₄-C₂₀) alkyl radical being derived from hydrogenated tallow), sold respectively under the names “Acylglutamate CS-11”, “Acylglutamate LS-11” and “Acylglutamate HS-21” by the company Ajinomoto. Mention may also be made of N-acyl lysines such as lauroyllysine sold under the name “Amihope LL” by the company Ajinomoto. The N-acyl amino acid derivatives and salts thereof are preferably N-acyl sarcosinates such as the sodium lauroylsarcosinate sold under the name “Oramix L30” by the company SEPPIC and the sodium myristoylsarcosinate and sodium palmitoylsarcosinate sold respectively under the names “Nikkol Sarcosinate MN”, and “Nikkol Sarcosinate PN” by the company Nikko Chemicals.

Among the N-acyl peptides which may be mentioned are those derived from all or part of collagen or keratin, such as the sodium lauryl collagen and palmitoyl keratin sold under the names “Proteol B 30” and “Lipacide PK” by the company SEPPIC.

(3) Among the alkyl or alkenyl ether or ester sulphates, derivatives thereof and salts thereof, the ones that are preferably used are those for which the alkyl or alkenyl radical contains at least 12 carbon atoms.

Among the alkyl or alkenyl ether sulphates, the ones that are preferably used are alkyl ether sulphate salts and in particular sodium lauryl ether sulphate. Among the alkyl or alkenyl ester sulphates which may be mentioned, for example, are isethionic acid esters and its salts, and in particular the sodium cocoyl isethionlate sold under the name “Geropon AC 78” by the company Rhône-Poulenc.

(4) Among the polyoxyethylenated fatty alkyl or alkenyl ethers or ester which are preferably used are those for which the alkyl or alkenyl radical contains at least 12 carbon atoms. Those particularly preferred contain at least 20 ethylene oxide units, such as, for example, PEG-20 stearate, laureth-23, oleth-20 and PEG-25 phytosterol.

(5) Among the polyoxyethylenated alkyl or alkenyl carboxylic acid and salts thereof which are preferably used are those including at least 10 ethylene oxide units, such as, for example, laureth-10 carboxylic acid and oleth-10 carboxylic acid.

(6) Among the N-alkyl or alkenyl betaines which are preferably used are those for which the alkyl or alkenyl radical contains at least 12 carbon atoms, such as, for example, laurylamidopropylbetaine and oleyl-amidopropylbetaine.

(7) Among the alkyl or alkenyl trimethylammoniums and salts thereof which are preferably used are those for which the alkyl or alkenyl radical contains at least 12 carbon atoms. Salts which are preferably used are the bromides and chlorides, such as cocoyltrimethylammonium chloride and cetyltrimethylammonium bromide.

When the compound (i) is an N-2-alkoxycarbonyl derivative of N-methylglucamine of formula (I), polyglyceryl-3 hydroxylauryl ether, sodium lauryl ether sulphate for cetyltrimethylammonium bromide is preferably used as dispersing and stabilizing agent (ii).

According to a second embodiment of the invention, the cubic gel particles are formed from a mixture of at last two amphiphilic compounds, one of the amphiphilic compounds being capable of forming a lamellar phase in the presence of water, and the other being capable of forming an inverse hexagonal phase in the presence of water.

The mixture of the two amphiphilic compounds forming the cubic gel particles is characterized in that neither of the two amphiphilic compounds can produce by itself a cubic phase when it is placed in contact with water and in that only their mixture gives such a phase, axed in that, moreover, one of the amphiphilic compounds is capable of forming a lamellar phase in the presence of water, while the other amphiphilic compound is capable of forming an inverse hexagonal phase in the presence of water.

The term “lamellar phase” (phase D according to Ekwall) means a liquid crystal phase with plane symmetry, including several amphiphilic bilayers arranged in parallel and separated by a liquid medium which is generally water.

The term “inverse hexagonal phase” (phase F according to Ekwall) means a liquid crystal phase corresponding to a hexagonal arrangement of parallel cylinders filled with a liquid medium which is generally water, separated by a hydrocarbon-based environment corresponding to the fatty chains of the amphiphile.

A more precise definition of these names is given in Ekwall (1968), Adv. Liq. Cryst. (Brown G. H., Ed.), Chap. 1, 14. Each of these phases has a characteristic texture under a polarized-light microscope, a more precise description of which may be found in Rosevear (1968), JSCC, 19, 581 and in Lachampt and Vila (1969), Revue Francaise des Corps Gras, No. 2, 87-111. The entire contents of each of these references is hereby incorporated in their entireties by reference.

The amphiphilic compound capable of forming a lamellar phase is preferably chosen from diglyceryl monoesters such as diglyceryl isostearate (Solvay) and diglyceryl monoleate (Rylo PG 29® sold by the company Danisco), along or as a mixture.

The amphiphilic compound capable of forming an inverse hexagonal phase is preferably chosen from diglyceryl mono, di- or triesters, aminopolyol carbamates and mixtures thereof. Diglyceryl mono-, di- or triesters which may be mentioned, for example, include diglycerol 2-decyltetradecanoate and diglyceryl 5 di/trioleate (TSED 396® sold by the company Danisco). Aminopolyol carbamates which may be mentioned, inter alia, include 3 N (2-decyltetradecyloxycarbonyl)amino-1,2-propanediol and N-2-dodecylhexadecyloxycarbonyl-N-methyl-D-glucamine. These aminopolyol carbamates are disclosed in EP-A-666 251, the entire contents of which are hereby incorporated by reference.

The mixture of the two types of amphiphilic compound preferably includes of from 10% to 90% by weight and better still from 15% to 85% by weight of at least one amphiphilic compound capable of forming a lamellar phase, and from 10% to 90% by weight and better still from 15% to 85% by weight of at least one amphiphilic compound capable of forming an inverse hexagonal phase, relative to the total weight of the mixture. These ranges include all values and subranges therebetween, including 12, 14, 20, 25, 30, 35, 40, 45, 50, 60, 70, 75, and 80% as appropriate for the respective compounds.

The ratio between the two types of amphiphilic compound depends on the compounds used, and a person skilled in the art will know how to determine the amount of a each type of compound to be used in order to obtain cubic gel particles.

More specifically, the mixtures constituting the cubic gel particles in the compositions of the invention are preferably prepared using the following combinations:

−55% to 75% by weight of diglyceryl isostearate and 25% to 45% by weight of diglyceryl 2-decyltetradecanoate;

−30% to 65% by weight of diglyceryl isostearate and 35% to 70% by weight of diglyceryl di/trioleate;

−75% to 85% by weight of diglyceryl isostearate and 15% to 25% by weight of 3-N-(2-decyltetradecyloxycarbonyl)amino-1,2-propanediol;

−55% to 75% by weight of diglyceryl isostearate and 25% to 45% by weight of N-2-dodecylhexadecyloxycarbonyl-N-methyl-D-glucamine;

−15% to 50% by weight of diglyceryl monooleate and 50% to 85% by weight of diglyceryl di/trioleate.

In this second embodiment, the mixture of compounds constituting the cubic gel particles is preferably made as an aqueous dispersion, and preferably in the presence of at least one dispersing and stabilizing agent, this agent possibly being chosen in particular from the compounds (ii) mentioned above for the first embodiment of the cubic gel particles. When it is present, the dispersing and stabilizing agent is used in an amount ranging, for example, from about 0.1% to 3% by weight relative to the total weight of the dispersion. This range includes all values and subranges therebetween, including 0.2, 0.3, 0.4, 0.5, 0.7, 0.9, 1, 1.5, 2 and 2.5%.

In the two embodiments of the cubic gel particles described above, a water-insoluble ionic amphiphilic lipid may be added to the aqueous dispersion containing these particles, preferably in an amount ranging from 0.0005% to 5% by weight and better still from 0.001% to 2% by weight relative to the total weight of the dispersion. These ranges include all values and subranges therebetween, including 0.005, 0.01, 0.05, 0.1, 1.1, 1.5, 2.1, 2.5, 3, 3.5 and 4%.

Among the water-insoluble ionic amphiphilic lipids which may be mentioned in particular are:

(i) phospholipids such as natural phospholipids, for instance soya lecithin or egg lecithin, chemically or enzymatically modified phospholipids, for instance hydrogenated lecithin or the sodium salt of phosphatidic acid, and synthetic phospholipids such as dipalmitoylphosphatidylcholine,

(ii) phosphoric esters of fatty acids and salts thereof, in particular the sodium and potassium salts thereof, such as the monocetyl phosphate sold under the name “Monafax 160/” by the company Mona, and the dimyristyl phosphate sold under the name “Mexoryl SY” by the company Chimex,

(iii) N-acyl derivatives of glutamic acid and salts thereof, such a the monosodium stearoylglutamate sold under the name “Acylglutamate HS 11” by the company Ajinomoto, and the mixture monosodium cocoyl(C₁₄-C₂₀) alkyl glutamate the C₁₄-C₂₀ alkyl radical being derived from hydrogenated tallow, sold under the name “Acylglutamate GS 11” by the company Ajinomoto, (iv) the sodium cetyl sulphate sold under the name “Nikkol SCS” by the company Nikko Chemicals, (v) the sodium cocoyl monoglyceride sulphate sold under the name “Nikko SGC 80 N” by the company Nikko Chemicals, and

(vi) water-insoluble quaternary ammonium derivatives such as behenyltrimethylammonium chloride, dilauryl-dimethylammonium chloride, distearyldimethylammonium chloride, 4,5-dihydro-1-methyl-2-(C₁₄-C₂₀)alkyl-1-(2-(C₁₄-C₂₀)alkylaminoethyl)imidazolium methyl sulphate, the C₁₄-C₂₀ alkyl radicals being derived from hydrogenated to low, sold under the name “Rewoquat W75H” by the company Rewo Chemische, dialkylhydroxyethylmethylammonium methyl sulphate whose alkyl radicals are derived from hydrogenated or unhydrogenated, tallow, sold under the name “Stepanquat VP 85” by the company Stepan, and “Quaternium-82” sold by the company SEPPIC under the name “Amonyl DM”.

The incorporation of these water-insoluble ionic amphiphilic lipids gives the cubic gel particles a surface charge which results in electrostatic repulsion between the particles.

The cubic gel particles as defined above have a size which mad be modified by the nature and concentration of the compounds of which they are made. These particles generally have a number-average size, measured using a BI 90 laser granulometer from the company Brookhaven Instruments Corporation, of about from 0.05 μm to about 1 μm and preferably less than or equal to 0.5 μm. These ranges include all values and subranges therebetween, including 0.075, 0.1, and 0.75 μm.

It is also possible to incorporate active compounds of various types into the cubic gel particles. In particular, the said particles may contain a hydrophilic or lipophilic active principle. Needless to say, by virtue of the specific structure of the cubic gel particles, it is possible to incorporate therein both hydrophilic and lipophilic active principles, even if these active principles are incompatible to a certain extent.

The compositions which are used according to the invention may in particular constitute cosmetic and dermatological compositions. For such an application, they contain a physiologically acceptable medium. The expression “physiologically acceptable medium” means herein a medium which is compatible with the skin, the lips, the scalp, the eyelashes, the eyes and/or the hair. This physiologically acceptable medium may more particularly include water and optionally of a physiologically acceptable organic solvent chosen, for example, from lower alcohols containing from 1 to 4 carbon atoms, for instance ethanol, isopropanol, propanol or butanol; polyethylene glycols containing from 6 to 80 ethylene oxides; polyols, for instance propylene glycol, isoprene glycol, butylene glycol, glycerol or sorbitol. The physiologically acceptable medium of the composition according to the invention has a pH which is compatible with the skin and which preferably ranges from 3 to 8 and better still from 5 to 7.

According to one preferred embodiment, the compositions used in the present invention also include an oily phase, which especially provides a sensation of comfort and softness when applied to the skin. The amount of oily phase may range, for example, from 2% to 40% by weight and preferably from 5% to 25% by weight relative to the total weight of the composition, the remainder of the composition including the aqueous phase containing phytanetriol or consisting of the cubic gel particles containing phytanetriol or containing the aqueous dispersion of cubic gel particles containing phytanetriol. These ranges include all values and subranges therebetween, including 3, 4, 7, 10, 15, 20, 30, and 35%.

When phytanetriol is in cubic gel particles, the weight ratio of the amphiphilic compounds constituting the particles of the cubic phase and of the oily phase preferably ranges from 0.02/1 to 1/1 and better still from 0.05/1 to 0.5/1. These ranges include all values and subranges therebetween, including 0.03/1, 0.04/1, 0.1/1, 0.2/1, 0.3/1, and 0.4/1.

As oils which may be used in the invention, mention may be made of mineral oils (liquid petroleum jelly), plant oils (liquid fraction of karite butter, sunflower oil or apricot kernel oil), animal oils (perhydrosqualene), synthetic oils (hydrogenated polyisobutene, isostearyl neopentanoate or isopropyl myristate), non-volatile or volatile silicone oils (cyclomethicones such as cyclopentasiloxane and cyclohexasiloxane) and fluoro oils (perfluoropolyethers). Fatty substances which may also be used are fatty alcohols, fatty acids and waxes. The oily phase of the emulsion may also contain gums such as silicone gums, resins and waxes.

The composition containing an oily phase may be in the form of a water-in-oil (W/O) or oil-in-water (O/W) emulsion. According to one preferred embodiment, it is in the form of an oil-in-water emulsion.

In a known manner, the compositions of the invention may also contain adjuvants that are common in the cosmetic or dermatological fields, such as hydrophilic or lipophilic gelling agents, hydrophilic or lipophilic active agents, preserving agents, antioxidants, solvents, fragrances, fillers, screening agents, bactericides, odor absorbers, dyestuffs and salts. The amounts of these various adjuvants are those that are conventionally used in the field under consideration, and, for example, from 0.01 to 10% relative to the total weight of the composition. Depending on their nature, these adjuvants may be introduced into the fatty phase, into the aqueous phase and/or into lipid spherules.

As active agents, the composition may in particular contain other anti-pollution active agents, such as sphingolipids (see EPA-O 577 718, incorporated in its entirety by reference), screening agents such as octocrylene and butylmethoxydibenzoylmethane; moisturizers such as polyols and in particular glycerol.

Gelling agents which may be mentioned, for example, are cellulose derivatives such as hydroxyethylcellulose and alkylhydroxyethylcelluloses such as cetylhydroxyethylcellulose; algal derivatives such as satiagum; natural gums such as tragacanth or guar gum; synthetic polymers such as carboxyvinyl polymers or copolymers and in particular those sold under the names Carbopol^{R} by the company Goodrich or Synthalen^{R} by the company 3V SA. The proportion of gelling agent preferably ranges from 0.1% to 2% relative to the total weight of the composition.

The compositions used according to the invention may be more or less fluid and may have the appearance of a white or colored cream, an ointment, a milk, a lotion, a serum, a paste or a mousse. They may optionally be applied to the skin in the form of an aerosol. They may also be in solid form and, for example, in the form of a stick.

Preferably, the compositions used according to the invention are obtained according to a preparation process including at least two steps. The first step generally includes preparing an aqueous dispersion of cubic gel particles, as defined above, by fragmentation, using a homogenizer, of the compounds as defined above and of water, optionally in the presence of water-insoluble ionic amphiphilic lipids and/or of hydrophilic and/or lipophilic active principles and/or of a dispersing and stabilizing agent as are defined above. The homogenizer may be of the rotor-stator type with a high shear rate, such as a Virtis® or Heidolph Diax 600® machine or a high-pressure homogenizer working at between 200 and 1 800 bar approximately (20 to 180 MPa).

Needless to say, it is possible to introduce, at this stage in the preparation of the aqueous dispersion of cubic gel particles, various additives and/or active principles into the aqueous phase. After formation of the cubic gel particles, the dispersing and stabilizing agent is generally outside the said particles.

The second step then generally includes adding to the said dispersion obtained an oily phase optionally containing certain lipophilic additives and/or active principles and in subjecting the mixture to a mechanical stirring which may be carried out in particular using a homogenizer of the same type as those defined above.

Various additives and/or active principles may also be introduced at this stage in the preparation. Moreover, when it is desired to prepare a gelled dispersion, in a third step, an aqueous solution containing a gelling agent is generally added to the mixture obtained after the second step.

The compositions used according to the invention may in particular constitute a care product and/or make-up product. They may be used in particular to protect the body against the effects of pollution, and/or to improve cell respiration and/or to reduce desquamation and/or to prevent the keratin materials, and in particular the skin, from becoming dull or dirty.

Thus, another subject of the invention includes a treatment process for protecting a keratin material against the effects of pollution, which includes applying to the keratin material a composition containing an effective amount of cubic gel particles in a physiologically acceptable medium.

A subject of the invention is also a treatment process for improving the cell respiration and/or for reducing desquamation and/or for preventing the keratin material from becoming dull and/or dirty, which includes applying to the keratin material a composition containing an effective amount of cubic gel particles in a physiologically acceptable medium.

### EXAMPLES

Having generally described this invention, a further understanding can be obtained by reference to certain specific examples which are provided herein for purposes of illustration only and are not intended to be limiting unless otherwise specified. The names are, depending on the case, the chemical names or CTFA (International Cosmetic Ingredient Dictionary and Handbook) names and the amounts are in percentages by weight, except where otherwise mentioned.

### Example 1

| Phase A: | |
|---|---|
| Phytanetriol | 3.92% |
| Cetyl phosphate (sold under the name | 0.08% |
| “Arlatone MAP160” by the company Uniqema) | |
| Water | 1.6% |
| Phase B: | |
| Polysorbate 40 (sold under the name | 1% |
| “Montanox 40 DF” by the company SEPPIC) | |
| (dispersant) | |
| Triethanolamine | 0.04% |
| Water | 55.96% |
| Preserving agent | 0.3% |
| Phase C: | |
| Hydrogenated polyisobutene | 7.8% |
| Cyclohexasiloxane | 11.6% |
| Isostearyl neopentanoate | 2.6% |
| Fragrance | 0.1% |
| Phase D: | |
| Cetylhydroxyethylcellulose (sold under the | 1% |
| name “Natrosol Plus Grade 330CS” by the | |
| company Hercules) | |
| Water | 14% |

Procedure

First Step

The compounds of phase A are mixed together at room temperature. Phase B is added to this mixture at room temperature. The mixture is then dispersed and homogenized at room temperature using an “UlraTurrax T50” homogenizer fitted with a 45F dispersion head, at 10 000 rpm for 30 minutes.

Second Step

The oily mixture of phase C is added to the aqueous dispersion of cubic gel particles obtained above. The mixture is then homogenized at room temperature using a high-pressure homogenizer, by 4 homogenization treatments at 600 bar.

Third Step

The preparation obtained in the second step is gelled using the mixture of phase D. The mixture is then homogenized at room temperature using a paddle homogenizer for 30 minutes. A stable homogeneous cream which can be applied easily to the skin and which protects it against pollution is obtained.

Example A (Comparative): Oil-in-water Emulsion

| | |
|---|---|
| Example A (comparative): Oil-in-water emulsion | |
| Hydrogenated polyisobutene | 7.8% |
| Cyclohexasiloxane | 11.6% |
| Isostearyl neopentanoate | 2.6% |
| Preserving agents | 0.5% |
| Xanthan gum | 0.6% |
| Polyacrylamide/C13-14 isoparaffin/laureth-7 | 2% |
| (Sepigel 305 sold by the company SEPPIC) | |
| Dimethicone copolyol (DC2-5695 from the | 3% |
| company Dow Corning) | |
| Cyclohexasiloxane | 11.7% |
| Glycerol | 3% |
| Water | qs 100% |
| Example B (comparative): Water-in-oil emulsion | |
| Hydrogenated polyisobutene | 7.8% |
| Cyclohexasiloxane | 11.7% |
| Isostearyl neopentanoate | 2.6% |
| Sodium chloride | 0.6% |
| Cetyldimethicone copolyol (Abil EM90 from | 3% |
| the company Goldschmidt) | |
| Glycerol | 3% |
| Water | qs 100% |

Test to Demonstrate in Vitro the Protective Effect of Cubic Gel Particles Anti-pollution Efficacy on Reconstructed Skin

The compositions of Example 1 and of Comparative Examples A and B were applied (2 mg/cm²) to the surface of reconstructed-skin epidermal samples and left in contact with them for 30 minutes at room temperature. Carbon-14 radio labeled particles were then applied to the epidermal samples and left in contact with them for 2 hours in the usual epidermal maintenance medium. Next, the epidermal samples were removed from their maintenance medium and washed several times with PBS buffer (phosphate-buffered saline). The washings allow weakly adsorbed particles to be removed from the epidermal samples without removing the composition initially applied. The levels of residual radio labeled particles were then evaluated by measuring the carbon-14 radioactivity added to the particles. The table below gives the results as percentages of residual particles relative to the amount of particles applied.

| | % relative to the amount of |
|---|---|
| | particles applied |
| Untreated area | 36.2 ± 2.83% |
| Area treated with composition of Example 1 | 3.3 ± 0.66 |
| Area treated with emulsion of Example A | 11.3 ± 0.65 |
| (comparative) | |
| Area treated with emulsion of Example B | 11.2 ± 2.10 |
| (comparative) | |

These results show that the compositions containing the cubic gel particles which are used according to the invention allow better protection of the skin against pollutant particles than conventional emulsions, by limiting the penetration of the external pollutant particles.

### Example 2

Fluid (O/W Emulsion)

According to the same procedure as for Example 1, a day fluid in the form of a dispersion was prepared by mixing together the following parts:

Phase A

| Phase A: | |
|---|---|
| Phytanetriol | 2.97% |
| Monosodium N-stearoylglutamate, (sold under | 0.03% |
| the name Acylglutamate HS-11 by the company | |
| Ajinomoto) | |
| Water | 1.25% |
| Phase B: | |
| Polysorbate 40 (sold under the name “Montanox | 0.75% |
| 40 DF° by the company SEPPIC) (dispersant) | |
| Glycerol | 4% |
| Propylene glycol | 4% |
| Water | 55.8% |
| Preserving agent | 0.2% |
| Phase C: | |
| Octocrylene | 8.4% |
| Butylmethoxydibenzoylmethane | 3.6% |
| Dimethicone (DC200 Fluid 1.5 cSt) | 4% |
| Isostearyl neopentanoate | 2% |
| Isopropyl myristate | 2% |
| Phase D: | |
| Preserving agent | 1% |
| Water | 10% |

A fluid composition is obtained, which may be applied in the form of a spray and which allows good protection of the skin.

### Example 3

Milk (O/W Emulsion)

According to the same procedure as in Example 1, a day fluid in the form of a dispersion was prepared by mixing together the following parts:

Phase A

| Phase A: | |
|---|---|
| Diglyceryl isostearate (Solway) | 1.8% |
| Cetyl phosphate (sold under the name | 0.03% |
| “Arlatone MAP 160011 by the company Uniqema) | |
| Diglyceryl 2-decyltetradecanoate | 1.2% |
| Water | 1.25% |
| Phase B: | |
| Triethanolamine | 0.01% |
| Glycerol | 3% |
| Water | 63% |
| Preserving agent | 0.3% |
| Phase C: | |
| Perhydrosqualene | 5% |
| Stearyl heptanoate/stearyl octanoate | 4% |
| Apricot kernel oil | 5% |
| Cyclohexasiloxane | 2% |
| Preserving agent | 0.1% |
| Phase D: | |
| Cetylhydroxyethylcellulose (sold under the | 0.5% |
| name “Polysurf 67” by the company Hercules) | |
| Water | 12.81% |

A milk which is pleasant to apply to the skin and which gives good protection against pollutants is obtained.

### Example 4

Day Cream (O/W Emulsion)

According to the same procedure as for Example 1, a day cream was prepared by mixing together the following parts:

| Phase A: | |
|---|---|
| Diglyceryl isostearate (Solway) | 1.2% |
| Diglyceryl di/trioleate (sold under the | 1.8% |
| name “TS-ED 396” by the company Danisco) | |
| Water | 1.25% |
| Phase B: | |
| Polysorbate 40 (sold under the name | 1% |
| “Montanox 40 DF” by the company SEPPIC) | |
| Glycerol | 3% |
| Water | 60.35% |
| Preserving agent | 0.3% |
| Phase C: | |
| Perhydrosqualene | 4% |
| Apricot kernel oil | 7% |
| Cyclopentasiloxane | 5% |
| Preserving agent | 0.1% |
| Phase D: | |
| Cetylhydroxyethylcellulose (sold under the | 1% |
| name “Natrosol Plus Grade 330CS” by the | |
| company Hercules) | |
| Water | 14% |

A comfortable day cream which is capable of effectively protecting the skin against pollution is obtained.

This application is based on French patent application 0007342, filed Jun. 8, 2000, the entire contents of which are hereby incorporated by reference, the same as if set forth at length.

Having now fully described this invention, it will be apparent to one of ordinary skill in the art that many changes and modifications can be made thereto without departing from the spirit or scope of the invention as set forth herein.

1. A method of protecting keratin materials from the harmful effects of pollution, comprising topically applying a composition comprising an effective amount of cubic gel particles to said keratin materials.

2. The method of claim 1, wherein said effective amount ranges from 0.1 to 20% by weight, based on the total weight of the composition.

3. The method of claim 1, wherein the cubic gel particles are in aqueous dispersion.

4. The method of claim 1, wherein the cubic gel particles are formed from a mixture comprising:
(i) 0.1% to 15% by weight, relative to the total weight of the composition, of at least one compound selected from the group consisting of 3,7,11,15-tetramethyl-1,2,3-hexadecanetriol, phytanetriol, N-2-alkoxycarbonyl derivatives of N-methylglucamine and unsaturated fatty acid monoglycerides; and
(ii) 0.05% to 3% by weight, relative to the total weight of the composition, of at least one dispersing and stabilizing agent selected from the group consisting of surfactants that are water-soluble at room temperature and containing a saturated or unsaturated, linear or branched fatty chain containing from 8 to 22 carbon atoms.

5. The method of claim 4, wherein a weight proportion of compound (i) to said dispersing and stabilizing agent (ii) ranges from 2 to 200.

6. The method of claim 4, wherein said N-2-alkoxycarbonyl derivative of N-methylglucamine corresponds to formula (I) below: 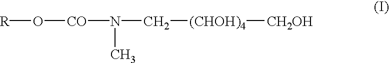
in which R represents a branched alkyl radical containing from 6 to 18 carbon atoms.

7. The method of claim 6, wherein said N-2-alkoxycarbonyl derivative of N-methylglucamine is chosen from N-2-hexyldecyloxycarbonyl-N-methylglucamine, N-2-ethyl-hexylcarbonyl-N-methylglucamine and N-2-butyloctyloxycarbonyl-N-methylglucamine, and mixtures thereof.

8. The method of claim 4, wherein the cubic gel particles contain as compound (i) a mixture consisting of from 1% to 40% by weight of phytanetriol relative to the weight of the mixture and from 60% to 99% by weight of N-2-alkoxycarbonyl derivative of N-methylglucamine relative to the weight of the mixture.

9. The method of claim 4, wherein said unsaturated fatty acid monoglyceride is selected from the group consisting of glyceryl monooleate and glyceryl monolinoleate.

10. The method of claim 4, wherein the cubic gel particles contain as compound (i) a mixture consisting of from 1% to 50% by weight of phytanetriol relative to the weight of the mixture and from 50% to 99% by weight of unsaturated fatty acid monoglyceride relative to the weight of the mixture.

11. The method of claim 4, wherein said dispersing and stabilizing agent is at least one selected from the group consisting of:
(1) alkyl or alkenyl ethers or esters of a polyol,
(2) N-acyl amino acids and derivatives thereof, and peptides N-acylated with an alkyl or alkenyl radical, and salts thereof,
(3) alkyl or alkenyl ether or ester sulphates, derivatives thereof and salts thereof,
(4) polyoxyethylenated fatty alkyl or alkenyl ethers or esters,
(5) polyoxyethylenated alkyl or alkenyl carboxylic acids and salts thereof,
(6) N-alkyl or alkenyl betaines,
(7) alkyl or alkenyl trimethylammoniums and salts thereof, and
(8) mixtures thereof.

12. The method of claim 1, wherein the cubic gel particles are formed from a mixture of at least two amphiphilic compounds, one of the amphiphilic compounds being capable of forming a lamellar phase in the presence of water, and the other being capable of forming an inverse hexagonal phase in the presence of water.

13. The method of claim 12, wherein the amphiphilic compound capable of forming a lamellar phase is selected from the group consisting of diglyceryl monoesters.

14. The method of claim 12, wherein the amphiphilic compound capable of forming an inverse hexagonal phase is selected from the group consisting of diglyceryl mono-, di- or triesters and aminopolyol carbamates, and mixtures thereof.

15. The method of claim 12, wherein the amphiphilic compound capable of forming a lamellar phase is selected from the group consisting of diglyceryl isostearate and diglyceryl monooleate, and mixtures thereof.

16. The method of claim 12, wherein the amphiphilic compound capable of forming an inverse hexagonal phase is selected from the group consisting of diglyceryl 2-decyltetradecanoate, diglyceryl di/trioleate, 3-N-(2-decyltetradecyloxycarbonyl)amino1, 2-propanediol and N-2-dodecylhexadecyloxycarbonyl-N-methyl-D-glucamine, and mixtures thereof.

17. The method of claim 12, wherein the mixture of the two amphiphilic compounds consists of from 10% to 90% by weight of the amphiphilic compound capable of forming a lamellar phase and from 10% to 90% by weight of the amphiphilic compound capable of forming an inverse hexagonal phase, relative to the total weight of the mixture.

18. The method of claim 12, wherein the mixture of the two amphiphilic compounds is selected from the group consisting of the following mixtures:
−55% to 75% by weight of diglyceryl isostearate and 25% to 45% by weight of diglyceryl 2-decyltetradecanoate;
−30% to 65% by weight of diglyceryl isostearate and 35% to 70% by weight of diglyceryl di/trioleate;
−75% to 85% by weight of diglyceryl isostearate and 15% to 25% by weight of 3-N-(2-decyltetradecyloxycarbonyl)amino-1, 2-propanediol;
−55% to 75% by weight of diglyceryl isostearate and 25% to 45% by weight of N-2-dodecylhexadecyloxcarbonyl-N-methyl-D-glucamine; and
−15% to 50% by weight of diglyceryl monooleate and 50% to 85% by weight of diglyceryl di/trioleate.

19. The method of claim 1, wherein the cubic gel particles have a size ranging from 0.05 um to 1 μm.

20. The method of claim 3, wherein the dispersion of cubic gel particles further comprises at least one water-insoluble ionic amphiphilic lipid.

21. The method of claim 20, wherein said water-insoluble ionic amphiphilic lipid is at least one selected from the group consisting of:
(i) phospholipids,
(ii) phosphoric esters of fatty acids,
(iii) water-insoluble N-aryl derivatives of glutamic acid and salts thereof,
(iv) sodium cetyl sulphate,
(v) sodium cocoylmonoglyceride sulphate, and
(vi) water-insoluble quaternary ammonium derivatives.

22. The method of claim 1, wherein the cubic gel particles further comprise at least one hydrophilic and/or lipophilic active principle.

23. The method of claim 1, wherein the cubic gel particles are present in an amount ranging from 0.1% to 10% by weight relative to the total weight of the composition.

24. A treatment process for protecting a keratin material against the effects of pollution, comprising applying to keratin material a composition comprising an effective amount of cubic gel particles in a physiologically acceptable medium.

25. A treatment process for improving the cell respiration and/or for reducing desquamation and/or for preventing keratin material from becoming dull and/or dirty, comprising applying to the keratin material a composition comprising an effective amount of cubic gel particles in a physiologically acceptable medium.

26. The process of claim 24, wherein said keratin material is the skin.

27. The process of claim 25, wherein said keratin material is the skin.
